(19) United States
(12) Patent Application Publication
(10) Pub. No.: US 2003/0106555 A1
(43) Pub. Date: Jun. 12, 2003

(54) Nasal filter and sampler

(76) Inventor: Euan Tovey, New South Wales (AU)

Correspondence Address: Nixon & Vanderhye, 8th Floor, 1100 North Glebe Road, Arlington, VA, 22201-4714, US

(21) Appl. No.: 10/204,710

(22) PCT Filed: Feb. 23, 2001
(86) PCT No.: PCT/AU01/00194

(30) Foreign Application Priority Data
Feb. 24, 2000 (AU) PQ 5821

(51) Int. Cl. A61M 16/00

(52) U.S. Cl. 128/205.27; 128/206.11

(57) ABSTRACT

Device (10) may be inserted in a nostril to filter and/or sample air inhaled or exhaled. Air inhaled through device (10) enters aperture (21) and exits through aperture (25). While passing through device (10) the airstream is deflected around deflector (16) and caused to encounter collector (19) extending inwardly from sidewall (18). A liquid or adhesive on collector (19) entraps particles, e.g. allergens, in the airstream, and can be subsequently removed for analysis. Alternatively collector (19) can be replaced by a reservoir for delivery of a pharmaceutical to a subject by inhalation.

### FIELD OF THE INVENTION

The present invention relates to a filter or sampler adapted to recover all or some fine particulate matter from air inhaled or exhaled by a human or other mammalian animal.

### BACKGROUND ART

The inhalation of various aeroallergens may cause asthma, rhinitis and other conditions in many people and animals. In order to understand the nature of the condition and possible treatments it is desirable to collect these aeroallergens from the inhaled air stream. In addition, such collection can be used as a prophylactic measure to prevent aeroallergens from entering the airways of a subject.

While there is strong epidemiological evidence associating exposure to aeroallergens to both sensitisation and symptoms at a communal level, and to a lesser extent at an individual level, the methods for estimating personal exposure to aeroallergens are poorly developed. The most common method is to measure allergen concentration in settled dust (collected by a vacuum cleaner) which functions as a source of aeroallergens. The method has, however, serious confounders such as the concentration of allergen and quantity of dust/unit area varying more than 10 fold at different sites within a room. There is, however no consistent data to directly show that such samples relate to individual personal exposure.

Others have attempted to measure aeroallergens on stationary filters using an air pump. With this method the amount of aeroallergen per time or volume differs markedly with degrees of dust disturbance and pump flow rates. Generally, measurement of settled and airborne dust correlate only weakly with one another, if at all. Outdoor allergen sources, such as fungal spores or fallen particles, are estimated and generalised with spoor traps.

One available method generally used to measure personal exposure is to use filters worn on the upper body. These were developed for occupational sampling, eg. for asbestos and coal. Although they have been used occasionally for allergens they cannot be widely applied. This application is limited by battery life, low flow rates, consequent small samples as well as the relatively high cost and noise of such sampling devices. Such filters may not reflect what is actually being inhaled for several reasons. Firstly, spatial distribution of allergenic particles differs over small distances. For instance, in bed the face is close to a typical allergen source and the allergen may not travel to a filter a half metre away. Secondly, the collection of particles onto a vertical filter surface with a low constant face velocity is significantly different front such a collection involving variable airflow into a person's nostrils. Variables include changes in flow between and within each cycle of respiration and with exercise, and the effects of thermal body drafts, movement and wind.

Airborne allergens are mainly carried by large particles, although this varies with both the allergen involved and the circumstances. Mite allergens are mainly carried by mite faeces (>90% allergen 10-40 μm particles); cat allergens with dander particles (70% associated with >3 μm particles); fungal allergens depend upon the species and maturity (3 to 90 μm); pollen depending upon the species (15 to 60 μm, mainly 20 to 30 μm). What is airborne is dynamic and changes with time; small particles, for instance, have lower settling speeds and remain airborne for longer.

The nose of humans and other mammalian species efficiently collects particles, such as dust, pollens and bacteria, onto the mucosa by a combination of turbulence and impaction. Efficiency is determined by particle velocity, angular velocity, mass, size and space of the particle and the route that the particle takes in the nose.

There have been reports (Pasricha J. S. & Abrol B. M. Ann. Allergy 1974;32:331-333; French Patent specifications 2536659, and 2504003; U.S. Pat. Nos. 4,401,117 and 5,117,820) of the insertion of a tube containing a filter such as a wire mesh sieve into the nose for the purpose of relief of inhalant allergy. The use of such a wire filter with a pore size capable of removing most particles associated with allergy (ie. those >5 μm diameter) could be expected to have high airflow resistance and to be uncomfortable to use. In addition, as such a filter becomes loaded with particles its resistance would increase, making it more difficult to use. In addition, particulate material collected by such filters would be more difficult to completely remove in an unadulterated (virgin) state and so would not be in a form suitable for subsequent direct analysis.

In the applicant's U.S. Pat. No. 5,787,884, a nasal and oral filter adapted to fit within a nostril or mouth of a subject is described. The filter in this document is adapted to enable the flow of air into a passage. Once in the passage, the air is channelled towards and around a centrally located collector such that at least some of the particles in the airflow are caused to impact the collector.

The present invention aims to provide an alternative to the filters or samplers defined by the prior art and to maximise the collection of particulate matter inhaled and in some instances exhaled, by a subject.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### DISCLOSURE OF THE INVENTION

In a first aspect, the present invention is a filter for air inhaled or exhaled by a subject, the filter including a body having a first end, at least a second end, a side wall and at least one collection means positioned adjacent the side wall, the side wall surrounding an air flow deflector means, the side wall and air flow deflector means defining therebetween a passage through the body with at least a portion of the collection means extending into the passage such that, during inhalation or exhalation, air is drawn into the passage and deflected by the air flow deflector means, so causing matter in the air to impact with the at least one collection means.

In one embodiment of the invention, the filter is adapted to fit within at least one nostril of a subject. Alternatively, the filter may be adapted to fit within the mouth of a subject.

In another embodiment, the filter includes two distinct collection means.

In a further embodiment, the collection means extends inwardly from the side wall into the passage of the body such that it substantially obstructs direct airflow through the passage. In a preferred embodiment, the collection means is attached to or is integral with the side wall and may be positioned adjacent the second end.

In a further embodiment of the invention, two collection means are disposed substantially opposite each other within the body of the filter.

The collection means is preferably removable from the filter to allow analysis of the matter impacted thereon.

In a further embodiment, the sidewall is cylindrical. In this embodiment, the at least one collection means may be circumferentially disposed around some or all of the cylindrical side wall.

The at least one collection means may be planar in configuration and/or made from an air-impermeable material.

In another embodiment, the collection means may include a strip, patch or other piece of material adapted to collect matter in the inhaled or exhaled air, the strip, patch or other material being readily removable from the filter. Alternatively, the entire collection means may be removable from the filter.

The collection means may also include a fluid entrapment means to enhance the entrapment of matter, the fluid being removable from the collection means to allow analysis of the particulate matter entrapped therein.

The collection means may further include a material that will retain and denature the impacted matter. This embodiment of the invention is particularly useful in situations where the filter is used prophylactically to prevent the entry of particular aero-allergens into the airways of a subject. Appropriate materials for the retention of matter include soft hydrophobic materials such as oils and greases, whereas denaturing agents include aqueous gels including tannic acid, alum, oxidising agents or detergents (for example SDS). Other means of collecting particulate matter on the surface of the collection means include coating the surface of the collection means with materials which will irreversibly and non-specifically bind soluble macromolecules extracted from the particles. Such materials include nitrocellulose, activated nylons, polyvinylidene fluoride (PVDF) and protein-binding gels.

In a further embodiment, the collection means may be coated with an adhesive or another substance that will enhance particle entrapment.

In another embodiment, at least a portion of the air flow deflector is made from a material that is gas absorbable. The material may be specific for a single gas such as nitric oxide. In this regard, it is known that the presence of nitric oxide is indicative of an inflammatory response in the airways, nitric oxide being released from the inflamed tissues in the exhaled air. The presence of a particular gas in the exhaled air may be measured by a later analysis of the air flow deflector means, for example, by gas chromatography. Additionally, this embodiment has the further feature of substantially preventing the inhalation of a particular gas in inhaled air as the gas is absorbed by the material of the air flow deflector when the inhaled air comes into contact with the air flow deflector means.

The air flow deflector means is preferably centrally located within the body of the filter.

In another embodiment, at least a portion of the air flow deflector means is semi-permeable. For example, at least a portion of the air flow deflector means can be made from an elastomeric or foam material.

The air flow deflector means may also be conical, hemispherical or pyramidal in shape.

In a further embodiment, at least a portion of the air flow deflector means is made from a coated material such that matter in the air that strikes the air flow deflector means is retained thereon.

In another embodiment, the air flow deflector means is made from a compressible material or a pleated material. The air flow deflector may further comprise a hollow shell having a thin laminar skin.

The air flow deflector means may also be supported within the body of the filter by strut members connecting the air flow deflector means to the side wall of the body. This embodiment has the advantage that when the filter is inserted into the nostril or mouth of a subject, the side wall will typically be forcibly deformed to some extend. Such deformation causes the struts to compress the air flow deflector means such that the airflow passage is retained in a substantially open configuration when the filter is in use.

In another embodiment, the strut members are air impermeable but define a channel through which inhaled air in the passage may flow.

In a further embodiment, the filter includes an inlet passage in fluid communication with the passage formed between the air flow deflector means and the side wall.

In another embodiment, the present invention provides a plurality of passages through the body of the filter. In this embodiment, each passage is formed from a tubular structure, each tubular structure having an inlet and an outlet. Each tubular structure may spiral around the air flow deflector means such that the inhaled air flow is caused to rotate in its travel through the filter, thereby increasing the turbulence of the air flow.

In a further embodiment, each tubular structure is angled at the inlet such that the cross-sectional area of the member is increased. This embodiment also has the additional advantage of limiting the view of the interior of the body when looking into the body's inlet.

In another embodiment, the tubular members are interconnected by hinge members. The hinge members may be flexible and therefore facilitate compression of the body of the filter whilst maintaining the cross-sectional diameter of the tubular members. In this way, the filter may be compressed or distorted to fit into different shaped noses while still maintaining an open tubular structure to allow the inhalation, and if desired exhalation, of air through the filter.

In a second aspect, the present invention provides a filter for air inhaled or exhaled by a subject, the filter including a body having a first end, at least a second end, a side wall, and at least one collection means positioned adjacent the side wall, the side wall surrounding an air flow deflector means having one or more vanes extending outwardly therefrom, the side wall and air flow deflector means defining therebetween a passage through the body with at least a portion of the collection means extending into the passage such that, during inhalation or exhalation, air is drawn into the passage and deflected by the air flow deflector means, the vanes causing rotation of the inhaled air around the air flow deflector means such that matter in the air impacts with the at least one collection means.

The vanes are adapted to extend out and away from the air flow deflector means towards the side wall of the filter. The vanes may further abut with the side wall of the filter and may form an integral structure with the side wall. Furthermore, the vanes may be fixed relative the air deflector means or may rotate around the air flow deflector means.

The extension of the vanes away from the body of the air flow deflector means increases the surface area of the air flow deflector means creating a fan-like effect with inhaled or exhaled air being caused to rotate around the air flow deflector means as it travels towards the collection means.

In another embodiment, the plurality of vanes are configured such that each vane forms a spiral around the air flow deflector means to enhance the rotation of the inhaled or exhaled airflow around the air flow deflector means.

In a further embodiment, each vane may comprise a plurality of spiral shaped members. It is envisaged that such a double spiral arrangement may be used as a stand alone nasal filter or sampler. In this embodiment, the rotation and turbulence of the inhaled airflow is greatly increased such that the inhaled air is channelled towards the collections means and additionally, matter in the inhaled or exhaled air is caused to impact with the walls of the spiral shaped members.

In a further embodiment, matter in the inhaled or exhaled air may be caused to impact with the plurality of vanes in addition to impacting with the collection means. In this embodiment, the vanes may be coated with an adhesive agent to enhance collection of the matter in the inhaled or exhaled air.

In a still further embodiment, the body of the filter is made of a resilient material selected from the group comprising soft plastics material, natural or synthetic rubber and a silicone material. Other suitable materials are envisaged. Alternatively, the body of the filter may be formed of a rigid material and provided with a resilient surface coating or layer. In such cases, the body may be surrounded by a resilient cuff of a foam material or an inflatable material.

In a further embodiment, the filter is adapted to fit within the nostril of a human or other mammalian animal. Preferably, the filter forms a hermetic seal with an inside surface of the nostril.

The filter may also be worn on the nose of a user and as such may extend outside and across the nostrils of a user or may be retained in the nose by a portion of the filter which is arranged to fit within a nostril and to resiliently engage an adjacent surface of a nostril into which the filter is positioned. The filter may also be retained on the nose by means of a clip to one or both of the alar of the nostrils, or to the columnella between the nostrils, or by means of an elastic attachment to or around the head of the user. In this embodiment, the filter may include a body with a collection means which, when the filter is in position, is located either just outside or inside a nostril of the subject, the filter further including means to form a seal with the nostril, an inhalation passage through the body of the filter to allow air to be inhaled through the filter and an exhalation passage to allow air to be exhaled through the filter.

In a further embodiment, the body of the filter may also be attached to an adhesive tape or fabric which may in turn be attached to the user such that the tape or fabric extends across the nose of the user. If filtering is to occur at both nostrils, two filters may be employed and attached to the adhesive tape or fabric prior to the attachment of the tape or fabric across the nose. The use of adhesive tape or fabric to support the filter has the advantage that the filter may be used by persons with a wider range of nostril sizes than would otherwise be possible. It is desirable, though not essential, that the filter or adhesive tape or fabric form a hermetic seal with the nose or nostril. The filters and adhesive tape or fabric are preferably clear or flesh coloured to be less obtrusive to a wearer's appearance.

The filter of the present invention is preferably used to capture particles of various origins including biological, organic or mineral. Examples of particles include pollens, microbiological material, dander, debris, dust of all sorts, asbestos fibres and spores. Such particles can then be removed from the filter for analysis.

In a further embodiment, the filter acts prophylactically to prevent or substantially prevent the entry of particles into the airways of a subject. Accordingly, in this embodiment of the invention, the filter may be used to prevent exposure to allergens thus reducing the symptoms of, for example, hay fever.

The principal use of a device according to the present invention is the capture of at least some of the potentially allergenic particles in air about to be inhaled by a wearer. Such collected particles could be subject to diagnostic procedures such as culture, antibody probing or nucleic acid analysis.

In one embodiment, separate inhalation and exhalation pathways are provided in the device to ensure that the hot and moist exhaled air does not pass over the relatively cool and dry collection means. If the exhaled air is allowed to pass over the collection means, condensation can form on the collection means which may affect the viability of the collected samples and therefore adversely affect their subsequent analysis.

Accordingly, in one embodiment air is inhaled by a subject through an inhalation passage in the nasal filter and exhaled through a separate exhalation passage. Preferably, the inhalation passage and exhalation passage share, at least in part, a common passage within the filter. This may be achieved by the use of one-way valves in the passage.

In another embodiment of the invention, the filter collects particles of a different nature in separate regions of the filter. For example, the filter may include a first collection means adapted to collect particles of a larger size and a second collection means adapted to collect particles of a smaller size.

The filter may be taken apart to facilitate recovery of collected matter. Alternatively, however, the collection means may be removed from the filter while the filter is in situ. In an example where the collection means comprises a body of fluid adapted to collect particulate matter, the fluid may be syringed from the filter.

In a third aspect, the present invention provides a device for the delivery of a pharmaceutical substance to the airways of a subject, the device including a body having at least one inlet, at least one outlet, a sidewall and at least one reservoir means positioned adjacent the sidewall, the sidewall surrounding an air flow deflector means, the sidewall and air flow deflector means defining therebetween a passage through the body with at least a portion of the reservoir means extending into the passage such that, during inhalation, air is drawn into the passage and deflected by the air flow deflector means so causing the flow of inhaled air to impact with the reservoir means which in turn causes a pharmaceutical substance in the reservoir to be released and passed through the outlet of the device and into the airways of the subject.

In a fourth aspect, the present invention provides a device for the delivery of a pharmaceutical substance to the airways of a subject, the device including a body having at least one inlet, at least one outlet and a passage through the body to allow air to be inhaled by the animal through the device, the passage having a non-linear path and including at least one reservoir means placed in the passage so that, during inhalation, air is drawn towards and impacts with the reservoir means causing a pharmaceutical substance in the reservoir to be released and passed through the outlet of the device and into the airways of the subject.

Both the devices of the third and the fourth aspects have the advantage that they enable the long term release of a particular pharmaceutical substance into the airways of a subject.

In one embodiment of the third or fourth aspect, the reservoir means is air impermeable such that inhaled air impacts with the reservoir and is forced through the outlet of the device.

In a further embodiment, the reservoir means is semi-permeable such that a proportion of the inhaled air passes through the reservoir means and into the airways of the subject. In this embodiment, the passage of the inhaled air through the semi-permeable reservoir means causes release of a pharmaceutical substance in the reservoir, the pharmaceutical substance being caused to move through the semi-permeable reservoir means and into the airways of a human or other mammalian animal.

In another embodiment, the pharmaceutical substance in the reservoir means is caused to be released by turbulence of the inhaled air at the site of the reservoir. Alternatively release of the pharmaceutical substance may result from the force of impaction of the inhaled air against the reservoir means.

In a further embodiment, the device is adapted such that a substantially greater amount of a pharmaceutical substance is released from the reservoir means during inhalation than during exhalation.

In a still further embodiment, the pharmaceutical substance released from the reservoir means include analgesics, anti-inflammatory drugs, antibiotics, vaccines, encapsulated DNA, steroids, nicotine, morphine and insulin.

In another embodiment, the reservoir includes materials, such as sugar trehalose, which act to stabilise the pharmaceutical substance held in the reservoir means. In addition, the reservoir contains materials such that when the humidity in the reservoir means increases during the use of the device, the materials become less able to retain the pharmaceutical substance and it is released from the reservoir means into the airstream.

In a fifth aspect, the present invention provides a filter for air inhaled or exhaled by a subject, the filter including a body having a first end, at least a second end, a side wall, and a non-linear airflow passage through the body from an orifice at the first end to an orifice at the second end, the side wall surrounding a first collection means positioned in the air flow passage such that, during inhalation or exhalation, air is drawn into the passage initially towards and then channelled past the first collection means so that at least a proportion of any matter in the inhaled or exhaled air is caused to impact the first collection means and to be retained thereon, said first collection means and an interior surface of the side wall defining therebetween a portion of the airflow passage through the body, said interior surface further having a second collection means positioned such that at least a proportion of any matter in the inhaled or exhaled air that is channelled past the first collection means is caused to impact the second collection means and be retained thereon.

In a preferred embodiment of this aspect, the first collection means is centrally positioned in the air flow passage. The first collection means may be a planar disk positioned such that initial airflow into the body is substantially normal to the plane of the disk. The disk may be supported by a tower which extends from a proximal end adjacent the first end of the body to a distal end located within the body of the filter.

The tower defines a passage for the flow of air and is preferably held in place by a supporting means positioned between the side wall of the body and the tower. The tower preferably comprises a substantially hollow, cylindrical wall which includes a plurality of orifices formed in the wall to allow airflow therethrough. Alternatively, the tower may be oval or rectangular in cross-section. The orifices may be positioned at or adjacent the distal end of the tower or equally disposed around the wall of the tower.

Preferably, the orifices are separated by struts.

In one embodiment, the struts extend in a direction away from the wall of the tower and towards the sidewall of the filter body. The struts may further be adapted to form a spiral structure around the tower such that air exiting the orifices is channelled along a spiral formed by the struts before impacting with the second collection means and exiting through the second end. Matter in the inhaled or exhaled air is therefore caused to impact with not only the collection means but also the struts and the second collection means on the sidewall before exiting the filter via the second end. This embodiment has the advantage that the spiralling flow of air creates a cyclone effect and maintains the residence time of matter in the air at the second collection means for longer than if normal impaction had occurred. Accordingly, if matter is held for longer at the second collection means, it is envisaged that sample efficiency is increased. Furthermore, the first collection means may extend outwardly towards the sidewall of the filter body thereby forming a roof-like structure over the spiral struts. In this particular embodiment, as inhaled or exhaled air is channelled along the spirals formed by the struts, matter in the air is caused to impact with the extended first collection means.

In another embodiment, the cross-sectional diameter of the passage decreases as it extends towards the sidewall of the filter body. In this embodiment, the relatively smaller diameter causes an increase in the velocity of air.

In a further embodiment, the filter is adapted such that particles of a relatively large size are collected on the first collection means and particles of a relatively smaller size are collected on the second collection means.

In another embodiment, the first collection means may comprise a strip of adhesive tape stretched over the distal end of the tower. In this embodiment, it is preferred that at least a region of the wall of the tower adjacent the distal end is biased outwardly towards the side wall of the filter such that tension is applied to the adhesive strip.

The side wall of the filter body preferably expands in diameter from an area proximate the orifices of the tower in a direction towards the second end of the filter body.

In a further embodiment, the body diameter expands to a maximum diameter adjacent the distal end of the tower and then gradually decreases in diameter towards the second end of the filter body. The diameter of the orifice at the first end of the filter body is preferably substantially equal to the diameter of the orifice at the second end of the filter body.

The second collection means is preferably disposed around the entire diameter of the interior surface of the wall of the body. The second collection means further extends from a position on the interior surface adjacent the orifices in the tower to the second end of the body.

The first end of the body preferably flares out to form an external flange. The flange preferably forms a seal with at least one nostril when the user inhales and preferably acts as a valve when the user exhales.

In one embodiment, the sidewall of the body can be formed from a relatively soft material, such as silicone, while the tower and the planar disk supporting the first collection means can be formed from a relatively hard material, such as polypropylene. In this embodiment, the sidewall of the body forms a relatively flexible outer part of the device which may be deformed or compressed to fit within a nostril of a user. Furthermore, this embodiment has the advantage that, if the sidewall of the body is deformed in one cross-sectional direction to enable the device to fit within a nostril, rather than obstructing the air flow passage, the sidewall of the body is caused to deform in a second cross-sectional direction thereby retaining the passage in a substantially open configuration.

In another embodiment, the filter can be formed in one piece from one material.

In a sixth aspect, the present invention provides a filter for air inhaled or exhaled by a subject, the filter including a body having a first end, at least a second end, a side wall, and a non-linear airflow passage through the body from an orifice at the first end to an orifice at the second ends the side wall surrounding a collection means positioned in the air flow passage such that, during inhalation or exhalation, air is drawn into a first portion of the passage towards the collection means so that at least a proportion of any matter in the inhaled or exhaled air is caused to impact the collection means and to be retained thereon, said collection means and an interior surface of the side wall defining therebetween a second portion of the airflow passage, the first portion of the airflow passage defined by a tower member, said tower member comprising a substantially hollow body having a first end extending from a region proximate the first end of the body to a distal end internal the body and wherein the distal end of the tower member supports the collection means.

In one embodiment of the sixth aspect, the sidewall of the body is formed from a relatively soft material while the tower member and the collection means is formed from a relatively hard material. In this embodiment, the sidewall of the body forms a relatively flexible outer part of the device which may be deformed or compressed to fit within a nostril of a user. Furthermore, this embodiment has the advantage that, if the sidewall of the body is deformed in one cross-sectional direction to enable the device to fit within a nostril, rather than obstructing the second portion of the airflow passages the sidewall of the body is caused to deform in a second cross-sectional direction thereby retaining the second portion of the airflow passage in a substantially open configuration.

The tower member may be cylindrical, oval or rectangular in cross section.

In a seventh aspect, the present invention provides a method for filtering the inhaled or exhaled air of a subject, said method including the steps of placing adjacent to or inserting into the mouth or a nostril of the subject, a filter according any one of the aspects of the present invention and causing the subject to inhale or exhale through the filter.

### BRIEF DESCRIPTION OF THE DRAWINGS

By way of example only, preferred embodiments of the invention are described with reference to the accompanying drawings, in which:

FIG. 1 is a partly cut away, schematic, side elevational view of the face of a person in each of whose nostrils has been placed a filter according to this invention. Also shown in phantom in FIG. 1 is a filter of the invention inserted into the mouth of a user.

FIG. 2a is a vertical cross sectional view through one embodiment of a filter according to the present invention.

FIG. 2b is a vertical cross sectional view through the embodiment depicted in FIG. 2a when positioned within a nostril of a user.

FIG. 3 is a schematic perspective view of the filter of FIG. 2 showing the passage through the filter. The outline of the body of the filter is shown for the purposes of clarity.

FIG. 4 is a cut-away vertical cross sectional view of one embodiment of a collection means in a filter according to the present invention.

FIG. 5 is a perspective view of one embodiment of an air flow deflector means for use in a filter according to the present invention.

FIG. 6 is a perspective view of another embodiment of the air flow deflector means.

FIG. 7 is a perspective view of yet another embodiment of the air flow deflector means.

FIG. 8 is a partly cut-away perspective view of one embodiment of a region of another filter according to the present invention.

FIG. 9 is a cross-sectional view through line I-I of FIG. 8.

FIG. 10 is a cross-sectional view through one embodiment of a pharmaceutical delivery means according to the present invention.

FIG. 11 is a cross-sectional view through a further embodiment a pharmaceutical delivery means.

FIG. 12 is a cross-sectional view through one embodiment of another aspect of the present invention.

FIG. 13 is a schematic perspective view of the embodiment depicted in FIG. 12.

FIG. 14 is a schematic perspective view of a filter of a further aspect of the present invention.

FIG. 15 is a longitudinal cross-sectional view of the aspect of the invention depicted in FIG. 14.

FIG. 16a is a side cross-sectional view of the aspect of the invention depicted in FIG. 14.

FIG. 16b is a cross sectional view through II-II of FIG. 16a.

FIG. 17 is a partly cut-away schematic view of a filter of another embodiment of the present invention.

FIG. 18 is a side cross-sectional view of the embodiment of the invention depicted in FIG. 17.

FIG. 19 is a cross-sectional view through I-I of FIG. 18.

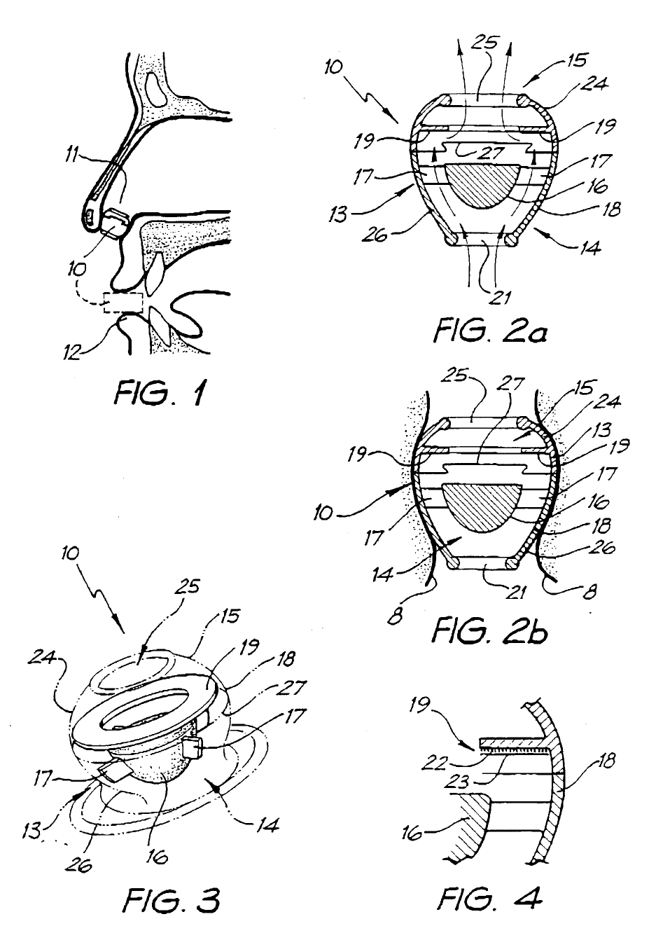

### DESCRIPTION OF THE INVENTION

The filter 10 of the present invention may be inserted into a nostril 11 or a mouth 12 of a user to filter and collect particles present in inhaled or exhaled air.

As depicted in FIGS. 2a and 2b, the filter 10 comprises a body 13 having two ends 14 and 15, the body 13 having an inlet 21 at end 14 to allow the flow of inhaled air into the body 13. An air flow deflector 16 is positioned centrally within the body 13 of the filter 10 and held in place by supports 17. A collection area 19 is positioned superior the deflector 16 and is connected to and extends inwardly from side wall 18 of the body 13.

In use, the filter 10 is inserted into the nostril 8 or mouth 9 of a user. The user breathes through the filter 10 for a period to collect a sample of inhaled aeroallergens. Upon inspiration, air is inhaled through inlet 21 in the body 13. The inhaled air is subsequently deflected by the deflector 16 such that it is channelled around the deflector 16 and caused to impact with the collection area 19. The flow of air is depicted in FIG. 2a by arrows.

The collection area 19 comprises a fine structure 22 as depicted in FIG. 4, which enables the collection area 19 to retain a liquid 23. The liquid 23 acts to entrap particles in the inhaled air which can subsequently be removed, if required, for analysis.

The body 13 of the filter 10 is formed of a resilient synthetic plastics material in two parts. An upper part 24 includes an outlet 25 and the collection area 19. A lower part 26 includes the inlet 21 and the deflector 16. The two parts 24 and 26 are resiliently retained together by interengaging flanges 27.

After a period of time, the aeroallergens trapped on the collection area 19 can be recovered by the removal of the filter 10 from the nostril 11 or mouth 12 of a user. The filter can then be separated into its two parts 24 and 26 and the collection area 19 exposed and the impacted particulate matter therein recovered for analysis.

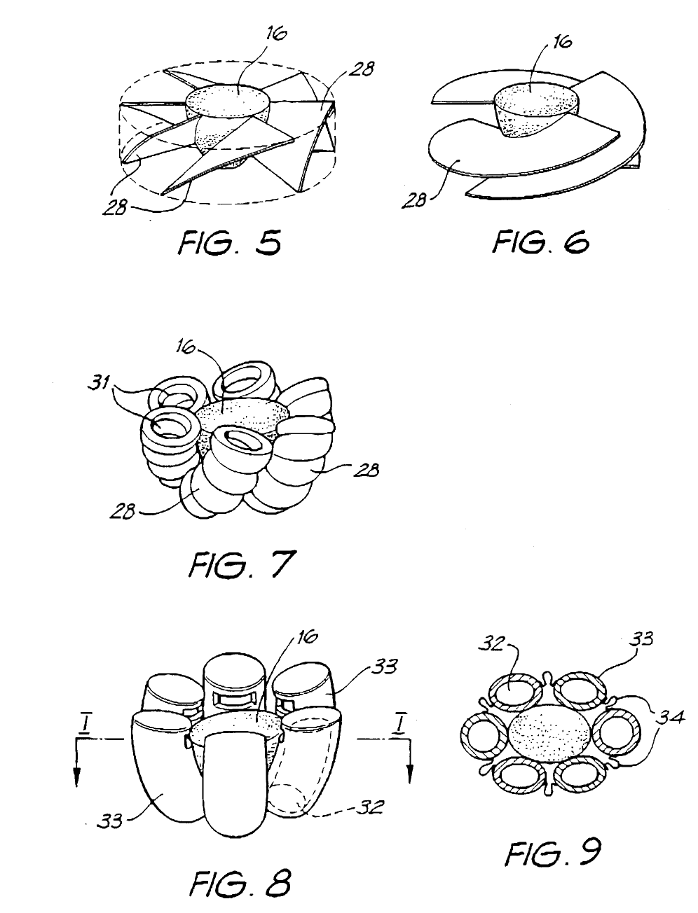

In one aspect, the deflector 16 includes a series of vanes 28 attached to the body of the deflector 16. This can be seen in FIG. 5, with further embodiments depicted in FIGS. 6 and 7. The vanes 28 may be adapted to extend towards the side wall 18 of the body 13 of the filter 10. In this way, the inhaled air is forced between the vanes 28 and towards the collection areas 19. In further embodiments, the vanes 28 may in fact abut with or be integral to the side wall 18 such that the vanes 28 act to replace the supports 17 which hold the deflector 16 in place within the body 13 of the filter 10 depicted in FIGS. 2 and 3. As depicted in FIG. 6, the vanes 28 may form a spiral around the deflector 16 such that any inhaled air is caused to rotate around the deflector 16, creating turbulence and generating centrifugal forces in the air flow. In a still further embodiment, as depicted in FIG. 7, each individual vane 28 may comprise a series of spirals 31, the entire vane 28 itself spiralling around the deflector 16.

Between the side wall 18 and the deflector 16, a passage 32 is formed, the passage 32 allowing the flow of air through the inlet and around the deflector 16. In one form as depicted in FIGS. 8 and 9, the passage comprises a series of tubes 33 arranged circumferentially between the deflector 16 and the side wall 18. The tubes 33 are connected to each other by interconnecting hinges 34, the hinges being of a compressible nature. In this way, the filter 10 may be compressed or distorted without compressing or distorting the tubes 33. Accordingly, the inflow of air into the filter is not compromised.

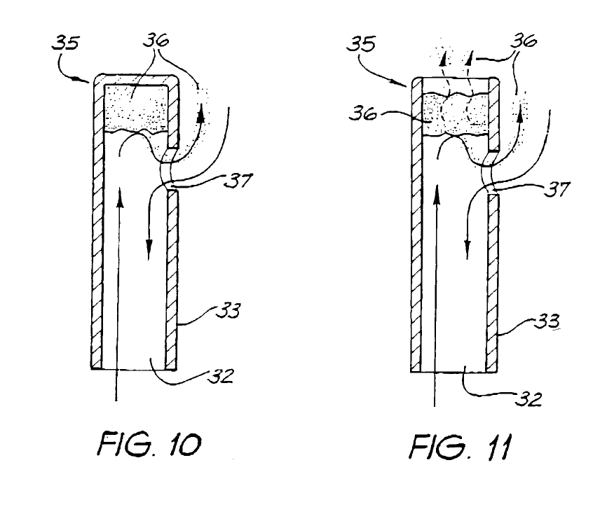

In another embodiment depicted in FIG. 10, the filter can have one or more tubes 33 is adapted such that it terminates in a reservoir 35, the reservoir 35 being adapted to contain a pharmaceutical substance 36 such as an analgesic. Located near or adjacent the reservoir is a gap 37 in the wall of the tube 33. Upon inhalation of air into the filter 10, the air is caused to move through the tube 33 such that it impacts with the reservoir 35. Impaction of the inhaled air with the reservoir 35 together with turbulence of the inhaled air around the reservoir 35 causes the pharmaceutical substance 36 to be released from the reservoir 35. The inhaled air together with the released pharmaceutical substance 36 then pass through the gap 37 and ultimately into the airways of the human or other mammalian animal in which the filter 10 is disposed. Alternatively, the reservoir may be made from a semi-permeable material such that the inhaled air moves through the reservoir 35 causing release of the pharmaceutical substance 36. In this form, a proportion of the inhaled air still passes through gap 37 and thus a proportion of the pharmaceutical substance 36 also passes through gap 37 in addition to passing through the reservoir 35.

As depicted in FIGS. 10 and 11, the likelihood of the pharmaceutical substance 36 being released from the reservoir 35 by the force of exhaled air is greatly reduced due to the reservoir 35 being located superior to the gap 37. In this way, the exhaled air is not directly caused to impact with the reservoir 35 and with this lack or at least decrease of impaction or turbulence, the pharmaceutical substance 36 will not likely be released from the reservoir 35 by exhaled airflow.

The reservoir 35 depicted in FIGS. 10 and 11 is sterically analogous to the collection area 19 and may be used to both collect particulate matter in inhaled air and act as a depot for a pharmaceutical substance 36.

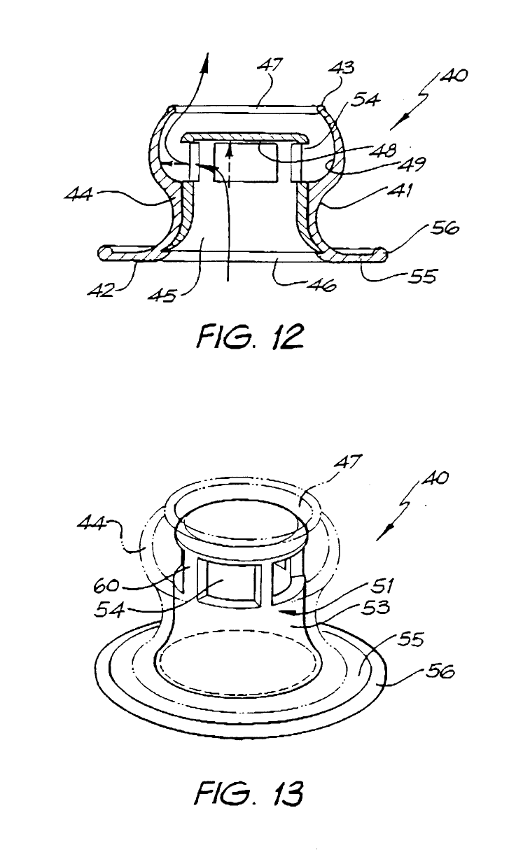

Another nasal filter according to the present invention is depicted generally as 40 in FIGS. 12 to 16. In this embodiment, the filter 40 comprises a body 41 having a first end 42, a second end 43, a side wall 44, and a non-linear airflow passage 45 that extends through the body 41 from an orifice 46 at the first end 42 to an orifice 47 at the second end 43.

The side wall 44 surrounds a planar first collection area 48 positioned normal to the air flow in the air flow passage 45 and centrally therein. During inhalation, air is drawn into the passage 45 initially towards and then channelled past the first collection area 48 so that at least a proportion of any particulate matter in the inhaled air is caused to impact the first collection area 48 and to be retained thereon.

The planar first collection area 48 and an interior surface of the side wall 44 define therebetween a portion of the airflow passage through the body. The interior surface has a second collection area 49 positioned such that at least a proportion of any particulate matter in the inhaled air that is channelled past the first collection area 48 is caused to impact the second collection area 49 and be retained thereon.

The planar collection area 48 is supported by a tower 51 extending into the body from the first end 42 of the body. The tower 51 has a substantially cylindrical wall 53 adjacent its distal end and gradually expands in diameter in region 52 towards the first end 42.

The wall of the tower 51 has a plurality of orifices 54 formed therein to allow airflow through the tower wall. The orifices 54 are preferably positioned at or adjacent the distal end of the tower 51.

The side wall 44 of the body 41 firstly expands to a maximum diameter adjacent the distal end of the tower 51 and then gradually decreases in diameter towards the second end 43. The diameter of the orifice 46 at the first end 42 of the body is substantially equal to the diameter of the orifice 47 at the second end 43.

The depicted second collection area 49 is disposed about the entire diameter of the interior surface of the wall 44 of the body 41. The second collection area 49 further extends from a position on the interior surface adjacent the orifices 54 in the tower 51 to the second end 43 of the body.

The first end 42 of the body expands radially to form a planar flange 55. The outer edge 56 of the planar flange 55 is adapted to form a seal with a nostril of a user of the filter 40 when the filter 40 is positioned in the nostril.

In the embodiment depicted in FIGS. 12 to 16, the sidewall 44 of the body is formed from a relatively soft and compressible silicone. The tower 51 and the first collection area 48 are formed from a relatively hard polypropylene material.

Both the first collection area 48 and the second collection area 49 may have the features of the collection areas described herein with reference to other filter embodiments.

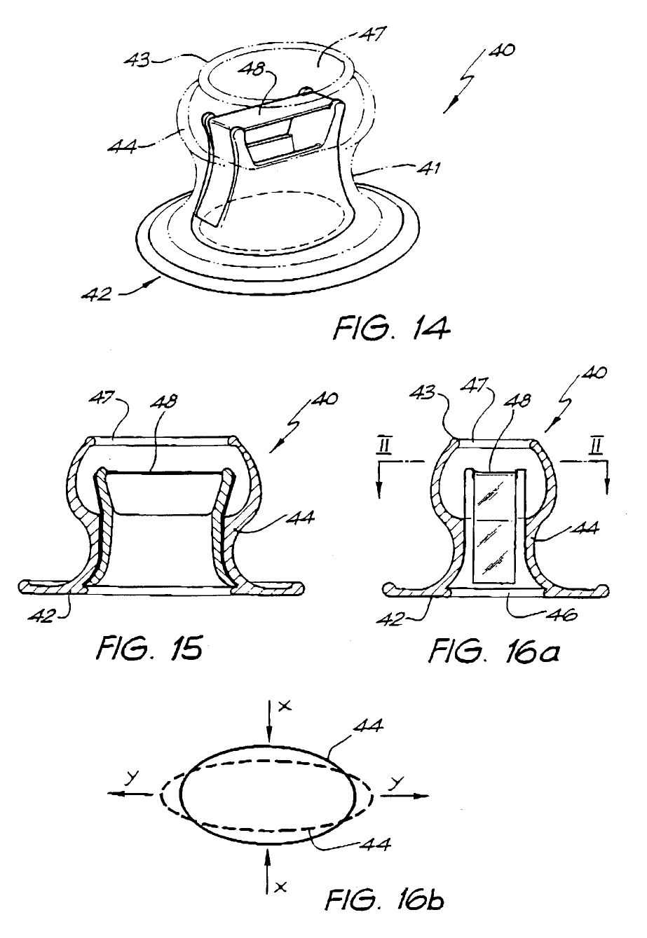

In the embodiment of the invention depicted in FIGS. 14 to 16, the first collection area 48 comprises a strip of adhesive tape stretched over the distal end of the tower 51. As depicted, it is preferred that at least a region of the cylindrical wall of the tower 51 adjacent the distal end is biased outwardly towards the side wall of the filter such that tension is applied to the adhesive strip.

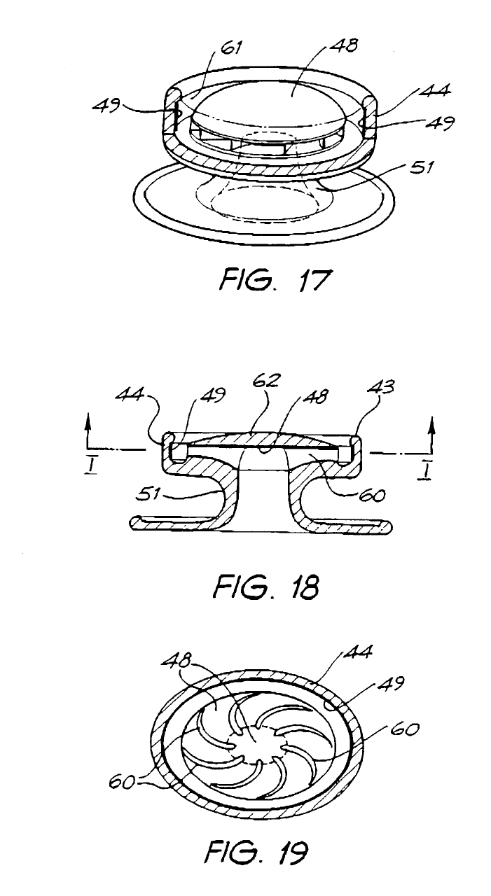

As depicted in FIGS. 12 to 16, the orifices 54 are separated by struts 60. In the embodiment of the invention depicted in FIGS. 17 to 19, the struts 60 extend in a direction away from the wall of the tower 51 and towards the sidewall 44 of the filter body. The struts 60 may further be adapted to form a spiral structure around the tower such that air exiting the orifices is channelled along a spiral formed by the struts before impacting with the second collection means located on the inner surface 61 of the wall 44. Matter in the inhaled or exhaled air is therefore caused to impact with not only the first collection area 48 but also the struts 60 and the second collection area 49 before exiting the filter 40 via the second end 43. This embodiment has the advantage that the spiralling flow of air creates a cyclone effect and maintains the residence time of matter in the air at the second collection area 49 for longer than if normal impaction had occurred. Accordingly, if matter is held for longer at the second collection area, it is envisaged that sample efficiency will be increased. Furthermore, the first collection area 48 may extend outwardly towards the sidewall of the filter body thereby forming a roof-like structure 62 over the struts 60. In this particular embodiment, as inhaled or exhaled air is channelled along the spirals formed by the struts, matter in the air is caused to impact with the roof-like structure 62.

As depicted in FIG. 16b, the sidewall 44 of the body 13 may be formed from a relatively soft material while the tower member 51 may be formed from a relatively hard material. In this embodiment, the sidewall 44 forms a relatively flexible outer part of the device which may be deformed or compressed to fit within a nostril 8 of a user. Furthermore, this embodiment has the advantage that, if the sidewall 44 of the body is deformed in one cross-sectional direction (x) to enable the device to fit within a nostril, rather than obstructing the airflow passage 32, the sidewall 44 of the body is caused to deform in a second cross-sectional direction (y) thereby retaining the airflow passage 34 in a substantially open configuration.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A filter for air inhaled or exhaled by a subject, the filter including a body having a first end, at least a second end, a side wall and at least one collection means positioned adjacent the side wall, the side wall surrounding an air flow deflector means, the side wall and air flow deflector means defining therebetween a passage through the body with at least a portion of the collection means extending into the passage such that, during inhalation or exhalation, air is drawn into the passage and deflected by the air flow deflector means, so causing matter in the air to impact with the at least one collection means.

2. The filter of claim 1 wherein the filter is adapted to fit within at least one nostril or a mouth of a subject.

3. The filter of claim 1 or claim 2 wherein the filter includes two separate collection means, each collection means located adjacent the side wall and disposed substantially opposite each other.

4. The filter of claims 1 or 2 wherein the sidewall is substantially cylindrical, oval or rectangular and the at least one collection means is circumferentially disposed around some or all of the side wall.

5. The filter of any one of the preceding claims wherein the at least one collection means is planar in configuration.

6. The filter of any one of the preceding claims wherein the at least one collection means is air-impermeable.

7. The filter of any one of the preceding claims wherein the at least one collection means is removable from the filter to facilitate analysis of the matter impacted thereon.

8. The filter of any one of the preceding claims wherein the at least one collection means is coated with an adhesive or another substance that will enhance entrapment of matter in the air.

9. The filter of any one of the preceding claims wherein the air flow deflector means is centrally located within the body of the filter.

10. The filter of any one of the preceding claims wherein the air flow deflector means is made from a compressible material.

11. The filter of claim 10 wherein the air flow deflector means is made from a foam material.

12. The filter of claim 10 wherein the air flow deflector means is a hollow structure.

13. The filter of any one of the preceding claims wherein at least a portion of the air flow deflector means is made from a gas absorbable material.

14. The filter of any one of the preceding claims wherein at least a portion of the air flow deflector means is made from a coated material such that matter in the air that strikes the air flow deflector means is retained thereon.

15. The filter of any one of the preceding claims wherein the air flow deflector means is connected to the side wall of the filter by strut members.

16. The filter of any one of the preceding claims wherein the body of the filter includes a plurality of passages therethrough.

17. The filter of claim 16 wherein each passage is formed from a tubular structure, each tubular structure having an inlet and an outlet.

18. The filter of claim 17 wherein each tubular structure forms a spiral around the air flow deflector means.

19. The filter of any one of the preceding claims further including a plurality of vanes, each vane extending from a proximal end located on the air flow deflector means to a distal end adjacent the side wall of the filter.

20. The filter of claim 19 or claim 20 wherein matter in the inhaled air impacts with a surface of each vane in addition to impacting with the at least one collection means.

21. The filter of any one of the preceding claims further including separate inhalation and exhalation passages.

22. A filter for air inhaled or exhaled by a subject, the filter including a body having a first end, at least a second end, a side wall, and at least one collection means positioned adjacent the side wall, the side wall surrounding an air flow deflector means having one or more vanes extending outwardly therefrom, the side wall and air flow deflector means defining therebetween a passage through the body with at least a portion of the collection means extending into the passage such that, during inhalation or exhalation, air is drawn into the passage and deflected by the air flow deflector means, the vanes causing rotation of the inhaled air around the air flow deflector means such that matter in the air impacts with the at least one collection means.

23. The filter of claim 22 wherein the vanes are fixed in position relative the air flow deflector means.

24. The filter of claim 22 wherein the plurality of vanes are capable of rotating about the air flow deflector means.

25. The filter of any one of claims 22 to 24 wherein each vane forms a spiral around the air flow deflector means.

26. The filter of any one of claims 22 to 25 wherein matter in the inhaled or exhaled air impacts with the plurality of vanes in addition to impacting with the collection means.

27. The filter of claim 26 wherein the vanes are coated with an adhesive agent to enhance collection of the matter in the inhaled or exhaled air.

28. A device for the delivery of a pharmaceutical substance to the airways of a subject, the device including a body having at least one inlet, at least one outlet, a sidewall and at least one reservoir means positioned adjacent the sidewall, the sidewall surrounding an air flow deflector means, the sidewall and air flow deflector means defining therebetween a passage through the body with at least a portion of the reservoir means extending into the passage such that, during inhalation air is drawn into the passage and deflected by the air flow deflector means so causing the flow of inhaled air to impact with the reservoir means causing a pharmaceutical substance in the reservoir to be released and passed through the outlet of the device and into the airways of the subject.

29. A device for the delivery of a pharmaceutical substance to the airways of a subject, the device including a body having at least one inlet, at least one outlet, and a passage through the body to allow air to be inhaled by the animal through the device, the passage having a non-linear path and including at least one reservoir means placed in the passage so that, during inhalation, air is drawn towards and impacts with the reservoir means causing a pharmaceutical substance in the reservoir to be released and passed through the outlet of the device and into the airways of the subject.

30. The device of claim 28 or claim 29 wherein the reservoir means is air impermeable.

31. The device of claim 28 or claim 29 wherein the reservoir means is semi-permeable.

32. The device of any one of claims 28 to 31 when used for the relatively long term release of a particular pharmaceutical substance into the airways of a subject.

33. The device of any one of claims 28 to 32 wherein the pharmaceutical substance in the reservoir means is caused to be released by turbulence of the inhaled air at the site of the reservoir.

34. The device of any one of claims 28 to 32 wherein the release of the pharmaceutical substance results from the force of impaction of the inhaled air against the reservoir means.

35. The device of any one of claims 28 to 34 wherein the pharmaceutical substance released from the reservoir means is selected from a group comprising analgesics, anti-inflammatory drugs, antibiotics, vaccines, encapsulated DNA, steroids, nicotine, morphine and insulin.

36. A filter for air inhaled or exhaled by a subject, the filter including a body having a first end, at least a second end, a side wall, and a non-linear airflow passage through the body from an orifice at the first end to an orifice at the second end, the side wall surrounding a first collection means positioned in the air flow passage such that, during inhalation or exhalation, air is drawn into the passage initially towards and then channelled past the first collection means so that at least a proportion of any matter in the inhaled or exhaled air is caused to impact the first collection means and to be retained thereon, said first collection means and an interior surface of the side wall defining therebetween a portion of the airflow passage through the body, said interior surface further having a second collection means positioned such that at least a proportion of any matter in the inhaled or exhaled air that is channelled past the first collection means is caused to impact the second collection means and be retained thereon.

37. The filter of claim 36 wherein the first collection means is centrally positioned in the air flow passage.

38. The filter of claim 36 or claim 37 wherein the first collection means is a planar disk positioned such that initial airflow into the body is substantially normal to the plane of the disk.

39. The filter of any one of claims 36 to 38 wherein the first collection means is supported by a tower member, said tower member extending from a proximal end adjacent the first end of the body to a distal end located within the body of the filter.

40. The filter of claim 39 wherein the tower member comprises a substantially hollow, cylindrical, oval or rectangular wall which defines a at last a portion of the airflow passage.

41. The filter of claim 40 wherein the tower member includes a plurality of orifices formed in the wall to allow airflow therethrough.

42. The filter of claim 41 wherein the orifices are separated by strut members.

43. The filter of claim 42 wherein the strut members extend in a direction away from the wall of the tower and towards the sidewall of the filter body.

44. The filter of claim 43 wherein the strut members form a spiral structure around the tower such that air exiting the orifices is channelled along a spiral formed by the struts before impacting with the second collection means and exiting through the second end.

45. The filter of any one of claims 36 to 44 wherein particulate matter is collected on the collection means and wherein further, the collection means collects particles of a relatively larger size to the particles collected on the second collection means.

46. The filter of any one of claims 39 to 45 wherein the first collection means comprises a strip of adhesive tape stretched over the distal end of the tower member.

47. The filter of claim 46 wherein at least a region of the wall of the tower adjacent the distal end is biased outwardly towards the side wall of the filter such that tension is applied to the adhesive strip.

48. The filter of any one of claims 36 to 47 wherein the first end of the body flares out to form an external flange, said flange forming a seal with at least one nostril when the user inhales and wherein further, the flange acts as a valve when the user exhales.

49. The filter of any one of claims 36 to 47 wherein the sidewall of the body is formed from a relatively soft material while the tower member is formed from a relatively hard material.

50. A filter for air inhaled or exhaled by a subject, the filter including a body having a first end, at least a second end, a side wall, and a non-linear airflow passage through the body from an orifice at the first end to an orifice at the second end, the side wall surrounding a collection means positioned in the air flow passage such that, during inhalation or exhalation, air is drawn into a first portion of the passage towards the collection means so that at least a proportion of any matter in the inhaled or exhaled air is caused to impact the collection means and to be retained thereon, said collection means and an interior surface of the side wall defining therebetween a second portion of the airflow passage, the first portion of the airflow passage defined by a tower member, said tower member comprising substantially cylindrical hollow body having a first end extending from a region proximate the first end of the body to a distal end internal the body and wherein the distal and of the tower member supports the collection means.

51. The filter of claim 50 wherein the sidewall of the body is formed from a soft material relative the material of the tower member and the collection means.

52. The filter of claim 51 wherein the relatively soft nature of the sidewall enables the filter to provide a good fit within the nostril of a user.

53. A method for filtering the inhaled or exhaled air of a subject, said method including the steps of placing adjacent to or inserting into the mouth or a nostril of the subject, a filter according any one of claims 1 to 27 and 36 to 52 and causing the subject to inhale or exhale through the filter.
